(19) United States
(12) Patent Application Publication
(10) Pub. No.: US 2005/0079139 A1
(43) Pub. Date: Apr. 14, 2005

(54) Minoxidil pharmaceutical foam formulation

(76) Inventors: Elizabeth Joan Jacques, Hool (GB); Stephen Tickle, Warrington (GB)

Correspondence Address: Daniel B. Schein, Ph.D., Esq., P.O. Box 28403, San Jose, CA, 95159, US

(21) Appl. No.: 10/962,292

(22) Filed: Oct. 8, 2004

(30) Foreign Application Priority Data
Oct. 11, 2003 (GB) 0323908.4

(51) Int. Cl. A61K 9/00; A61K 7/00

(52) U.S. Cl. 424/47; 514/269

(57) ABSTRACT

A pharmaceutical foam formulation in a dosage form which comprises an active ingredient selected from the group consisting of minoxidil, minoxidil sulphate, other soluble minoxidil salts, and mixtures thereof. An embodiment also includes a surfactant, water, and optional further excipients. In an embodiment, the formulation is adapted to form a foam when administered by spraying.

### FIELD OF THE INVENTION

This invention relates to a formulation of the pharmaceutical substance minoxidil and derivatives thereof.

### BACKGROUND OF THE INVENTION

Minoxidil is used for treatment of hereditary hair loss. Previously known formulations comprise a lotion applied using a dropper or by a roller to a target skin surface. Minoxidil is sparingly soluble in water and current products comprise propylene glycol, ethanol and water. Such formulations are awkward to administer and thorough coverage of the area to be treated can be difficult.

### SUMMARY OF THE INVENTION

A first aspect of the present invention is a pharmaceutical formulation comprising an active ingredient selected from the group consisting of:
minoxidil, minoxidil sulphate, other soluble minoxidil salts and mixtures thereof;
a surfactant;
water; and
optional further excipients.

Preferably the formulation is adapted to form a foam when administered by spraying.

### DESCRIPTION OF THE FIGURE

FIG. 1 is a chart of weights plotted against time to give a comparison of different foam qualities, wherein the weights are derived from a drip test over time so that firmer foam would drip less and hence weigh less.

### FURTHER DETAILS OF THE INVENTION

Percentages and amounts referred to in the specification are by weight unless stated otherwise. Percentages of the aforesaid ingredients are preferably selected to total 100%.

Dosage forms in accordance with this invention confer several advantages. The absence of ethanol and aerosol propellants reduces the risk of flammability. Ethanol can be an irritant to a user's skin. The absence of halocarbon aerosol propellants is beneficial to the environment.

The active ingredient is present in an effective amount. Preferably, the amount of active ingredient is about 2-5% by weight.

Preferably the amount of surfactant is about 2-10%. Any combination of anionic, cationic, non-ionic or amphoteric surfactants and non-ionic block copolymers may be used. Preferably ethoxylated glycerides, ethoxylated sorbitan esters, polyethoxylated and/or hydrogenated castor oil, nonionic block copolymers and amphoteric surfactants may be used.

Especially preferred surfactants may be selected from:
PEG 40 hydrogenated castor oil;
Polysorbate 20;
Cocamidopropyl betaine;
Glyceryl cocoate;
PEG 6 caprylic/capric glycerides, and
Poloxamer F 68.

Preferred formulations in which the active ingredient is minoxidil base include propylene glycol. An amount of about 50-80% is preferred. Propylene glycol is not essential when the active ingredient is minoxidil sulphate or other salt.

Preferred formulations include about 20-30% water.

Formulations in accordance with this invention produce a quick breaking foam when sprayed through a fine mesh or nozzle. This allows the foam to be rubbed in easily. The concentration of the active ingredient is selected to allow an effective amount to be administered in a volume of about 0.2-1.0 cm³, preferably 0.3-0.6 cm³, more preferably about 0.45 cm³ of the formulation solution.

Preferred formulations include buffers, pH stabilizers, emollients, skin conditioning agents and other excipients known to those skilled in the art.

According to the second aspect of the invention, there is provided a spray apparatus comprising a reservoir, metered dose valve and a dosage form in accordance with the first aspect of the present invention.

The invention is further described by means of example, but not in any limitative sense.

### EXAMPLE 1

A formulation containing minoxidil base was prepared using the following ingredients:

| | % w/v |
|---|---|
| Minoxidil | 2.0 |
| Propylene Glycol | 70.0 |
| Preservative | qs (quantum sufficit) |
| Cocamidopropyl betaine | 4.0 as 50% solution |
| Water | to 100 ml |

### EXAMPLE 2

A formulation containing minoxidil sulphate was prepared using the following ingredients:

| | % w/v |
|---|---|
| Minoxidil sulphate | 2.766 equal to 2.0% as base |
| Preservative | qs |
| Cocamidopropyl betaine | 2.0 as 50% solution |
| Water | to 100 ml |

### EXAMPLE 3

Test Method For Evaluation of Foam Quality

Equipment
Analytical top pan balance—five place with top opening.
Clean 20 ml syringe.
Clamp and clamp stand.
2 clean beakers—one of which must be at least 150 ml volume.
Stopwatch.

Method
1. Position the clamp stand adjacent to the balance.
2. Open the balance top and position the syringe in the clamp directly over the balance pan.
3. Place a clean empty beaker on the balance pan and either tare the beaker, or record the weight.
4. Take the clean 150 ml beaker and pump at least 100 ml of foam into it.
5. Immediately draw up the foam into the syringe, remove the plunger so that the barrel is full.
6. Immediately place the syringe in the clamp over the tared beaker.
7. Start the stop watch and record the foam/solution that drips from the syringe every minute for a minimum of five minutes.

The weights were plotted against time to give a comparison of foam qualities. The lower the solution weights were indicative of firmer foams.

The foam quality was evaluated gravimetrically and it was found that the cocamidopropyl betaine, polysorbate 20 and glyceryl cocoate produced firmer longer lasting foams. The PEG 6 caprylic/capric glycerides, poloxamer F68 and PEG 40 hydrogenated castor oil produced quick breaking foams.

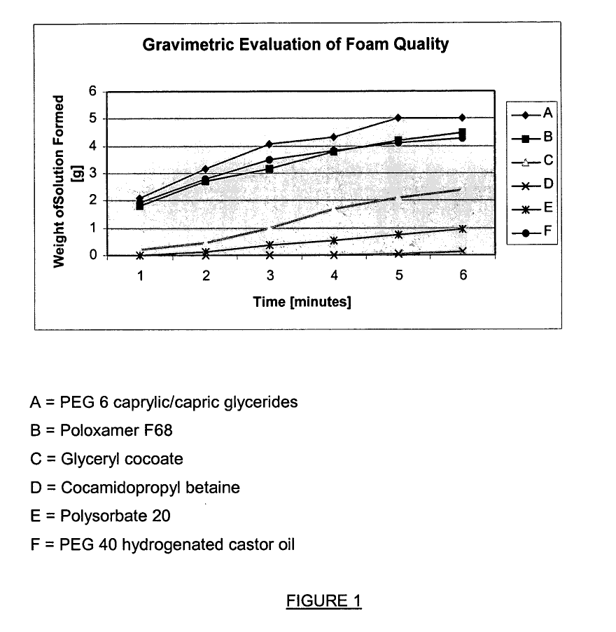

The results are shown in FIG. 1.

The product was found to be comparable with the current market products.

While preferred embodiments of the invention have been illustrated and described in detail in the FIGURE and foregoing description, the same is to be considered as illustrative and not restrictive in character. Thus, it is understood that all changes and modifications that come within the spirit of the invention are desired to be protected.

1. A pharmaceutical formulation, comprising at least one active ingredient selected from the group consisting of minoxidil, minoxidil sulphate, and a soluble minoxidil salt other than minoxidil sulphate,
a surfactant; and
water;
said formulation being adapted to form a foam when administered by spraying.

2. The formulation of claim 1, wherein the amount of said surfactant is about 2 to 10% by weight.

3. The formulation of claim 1, wherein said surfactant comprises at least one surfactant selected from the group consisting of an anionic surfactant, a cationic surfactant, a non-ionic surfactant, an amphoteric surfactant, and non-ionic block copolymers.

4. The formulation of claim 2, wherein said surfactant comprises at least one surfactant selected from the group consisting of an anionic surfactant, a cationic surfactant, a non-ionic surfactant, an amphoteric surfactant, and non-ionic block copolymers.

5. The formulation of claim 3, wherein said surfactant is selected from the group consisting of ethoxylated glycerides, ethoxylated sorbitan esters, polyethoxylated and hydrogenated castor oil, non-ionic block copolymers and amphoteric surfactants.

6. The formulation of claim 4, wherein said surfactant is selected from the group consisting of ethoxylated glycerides, ethoxylated sorbitan esters, polyethoxylated and hydrogenated castor oil, non-ionic block copolymers and amphoteric surfactants.

7. The formulation of claim 1, wherein said surfactant is selected from the group consisting of PEG 40 hydrogenated castor oil, Polysorbate 20, cocamidopropyl betaine, glyceryl cocoate, PEG 6 caprylic/capric glycerides and Poloxmer F68.

8. The formulation of claim 2, wherein said surfactant is selected from the group consisting of PEG 40 hydrogenated castor oil, Polysorbate 20, cocamidopropyl betaine, glyceryl cocoate, PEG 6 caprylic/capric glycerides and Poloxmer F68.

9. The formulation of claim 1, wherein when said active ingredient is minoxidil base, said formulation comprises about 50% to about 80% propylene glycol.

10. A method for administering minoxidil or a salt thereof, consisting of forming a foam containing minoxidil or a salt thereof.

11. The method of claim 10, wherein said foam is formed by spraying through a mesh or nozzle and said foam contains an effective amount of said active ingredient in a volume of about 0.2 cm³ to about 1.0 cm³.

12. The method of claim 11, wherein said foam volume is about 0.3 cm³ to about 0.6 cm³.

13. The method of claim 11, wherein said foam volume is about 0.45 cm³.

14. A spray apparatus for administering minoxidil or a salt thereof, comprising a reservoir, a metered dose valve, and a pharmaceutical formulation comprising at least one active ingredient selected from the group consisting of minoxidil, minoxidil sulphate, and a soluble minoxidil salt other than minoxidil sulphate,
a surfactant; and
water;
said formulation being adapted to form a foam when administered by spraying from said apparatus.

15. The apparatus of claim 14, wherein the amount of said surfactant is about 2 to 10% by weight.

16. The apparatus of claim 15, wherein said surfactant comprises at least one surfactant selected from the group consisting of an anionic surfactant, a cationic surfactant, a non-ionic surfactant, an amphoteric surfactant, and non-ionic block copolymers.

17. The apparatus of claim 15, wherein foam dispensed from said apparatus contains an effective amount of said active ingredient in a volume of about 0.2 cm³to about 1.0 cm³.

18. The formulation of claim 17, wherein when said active ingredient is minoxidil base, and said formulation comprises about 50% to about 80% propylene glycol.

19. The apparatus of claim 15, wherein said surfactant is selected from the group consisting of PEG 40 hydrogenated castor oil, Polysorbate 20, cocamidopropyl betaine, glyceryl cocoate, PEG 6 caprylic/capric glycerides, and Poloxmer F68.

20. The apparatus of claim 17, wherein said surfactant is selected from the group consisting of PEG 40 hydrogenated castor oil, Polysorbate 20, cocamidopropyl betaine, glyceryl cocoate, PEG 6 caprylic/capric glycerides and Poloxmer F68.
